(19) United States
(12) Patent Application Publication
(10) Pub. No.: US 2004/0249227 A1
(43) Pub. Date: Dec. 9, 2004

(54) Biosensor and method for detecting analytes by means of time-resolved luminescene

(76) Inventors: Holger Klapproth, Freiburg (DE); Mirko Lehmann, Freiburg (DE)

Correspondence Address: GRIFFIN & SZIPL, PC, SUITE PH-1, 2300 NINTH STREET, SOUTH, ARLINGTON, VA, 22204, US

(21) Appl. No.: 10/484,257

(22) PCT Filed: Jul. 18, 2002
(86) PCT No.: PCT/EP02/08021

(30) Foreign Application Priority Data
Jul. 18, 2001 (DE) 101 33 844.9

(51) Int. Cl. C07C 5/00

(52) U.S. Cl. 585/250

(57) ABSTRACT

The present invention concerns in general a biosensor in the form of a microchip for the optical detection of analytes and a method using this biosensor. In particular the invention concerns biosensors for detecting an analyte by time-resolved luminescence measurement and a corresponding method.

The present invention concerns in general a biosensor in the form of a microchip for the optical detection of analytes and a method using this biosensor. In particular the invention concerns biosensors for detecting an analyte by time-resolved luminescence measurement and a corresponding method.

### THE STATE OF THE ART

The use of essentially planar systems, described among experts as biosensors or biochips, i.e. biosensors in the form of microchips, for the qualitative and/or quantitative detection of the presence of certain substances, e.g. biomolecules, in a sample to be analysed, is already known. These biochips comprise a support on whose surface as a rule there is constructed a plurality of detection fields, for the most part in a matrix arrangement, whereby the individual fields or regions and/or groups of regions each differ from one another in their specificity towards a particular analyte that is to be detected. In the case where DNA analytes are to be detected, specific nucleic acid probes such as, for example, oligonucleotides or cDNA, for the most part in single-stranded form, whose individual specificity towards the nucleic acid to be detected is predetermined essentially by their sequence progression (probe design) are located, directly or indirectly immobilised, within the individual regions of the support surface. The microchip surface functionalised in this way is brought into contact with the sample that possibly contains the DNA analytes to be detected, in the context of an appropriate detection method, under conditions that ensure, in the event that the previously detectably labelled target nucleic acid(s) is/are present, that they will hybridise with the immobilised probe molecules. The qualitative and if necessary quantitative detection of one or more hybridisation complexes that are formed specifically takes place afterwards, in most cases by optical luminescence measurement and the assignment of the data that is obtained to the respective detection fields, thus enabling the determination of the presence of the DNA analyte(s) in the sample and if necessary their quantification.

It is known that this technology can also be used to detect other detectably labelled analytes, especially proteinaceous substances (peptides, proteins, antibodies and their functional fragments), provided that the detection reaction is based on the measurement of luminescence data. For example it is known that the amino-acid tyrosine shows a characteristic fluorescence whose half-life after excitation at approx. 260 nm enables use in accordance with the invention, and this even without the additional labelling of a proteinaceous substance having a tyrosine radical. Thus the use of peptides as collector molecules enables the detection of proteinaceous substances, e.g. antibodies or their fragments, as analytes, and this even without previously labelling the latter with a suitable luminophore.

In other words this technology enables any luminescence-based detection of a complex of a detectably labelled analyte (component from the sample to be analysed) and a collector molecule (immobilised support component) to be carried out, including even those systems in which the analyte is already characterized by a detectable intrinsic fluorescence and therefore does not need any further labelling.

Moreover, this technology can be applied to the measurement of pollutants such as polycyclic hydrocarbons or other organic substances. It is known that numerous representatives of the polycyclic hydrocarbons group show a fluorescence half-life of up to 450 ns and accordingly can be chosen as analytes even without additional labelling (for example pyrene with excitation at 336 nm). Thus these polycyclic hydrocarbons can be detected by the fact that they bond as analytes to antibodies produced specifically as collector molecules and yield a luminescence after suitable excitation.

In addition to the actual biochip or sensor chip, the systems based on luminescence detection and known in the prior art comprise in particular devices to acquire, convey and evaluate the luminescence signals. However, the products on the market are relatively expensive because of the large number of system components required together with the high complexity associated therewith, and essentially cannot be miniaturised any further.

WO 99/27 140 describes a biosensor in the form of a microchip comprising built-in detectors and optionally a built-in excitation source that is used to detect numerous biological analytes by means of luminescence measurement. This document teaches parallel excitation and measurement in each case of luminescence measurement. The unavoidable result of this is that a wavelength filter is interposed on the biosensor between the surface on which the luminophore is immobilised and the detector in order to mask out the excitation light and to enable the emitted luminescence light to be detected selectively. This filter that is compulsorily provided reduces the light yield and/or makes the manufacture of the biosensor more costly.

Therefore the task of the present invention is to provide novel biosensors of the aforementioned type with which the disadvantages of the systems known in the prior art are overcome.

A further task of the invention consists of being able to provide a more sensitive method to detect and/or determine one or more analytes in a sample that is assumed to contain the latter.

### SUMMARY OF THE INVENTION

The task of the invention is solved by an optical biosensor in the form of a microchip to detect a collector/analyte complex by means of luminescence, the biosensor comprising (a) a support with a surface onto which at least one type of collector molecule is immobilised, (b) at least one, preferably several detector(s) that can detect light passing through the surface, and (c) optionally at least one excitation source that can induce the emission of luminescence light, wherein the surface is the detector measuring surface or a surface of a layer arranged above the detector without an interposed wavelength filter for light from the excitation source or excitation wavelengths.

Preferably the biosensor comprises one or more excitation source(s) that can induce a luminophore to emit luminescence light and is/are most preferably integrated into the biosensor.

According to a preferred embodiment, the microchip is of monolithic design and the detector(s) is/are integrated into the support. Alternatively, the detector(s) in the form of a film can be attached to the support by adhesive.

Alternatively the detector(s) can be located close to the surface but if necessary at a distance away from the latter. Most preferably the distance between the surface (=place where the signal originates/luminescence light is emitted) and the measuring surface of the detector (=place where the signal is detected) is not greater than 10 μm, more preferably not greater than 5 μm and most preferably not greater than 1 μm.

In a preferred embodiment, the at least one type of collector molecule is immobilised onto the surface in individual detection fields or in the form of a matrix. More preferably, several types of collector molecules are immobilised onto the surface. Most preferably, different types of collector molecules are immobilised onto different detection fields or distinct positions of the matrix.

Preferably the collector molecules are selected from the group consisting of single or double stranded nucleic acids, nucleic acid analogues, haptenes, proteins, peptides, antibodies or their fragments, sugar structures, receptors or ligands.

According to a preferred embodiment, the biosensor of the invention can comprise in addition one or more elements from the group consisting of a control unit, at least one amplifier, one or more signal transformers, one or more storage/memory units, one or more filters, an optics system, light guides (optical fibres) and one or more protective layers; this subject to the proviso that no wavelength filter for light from the excitation source or the excitation wavelength is arranged or interposed between the detector(s) and the surface of the support onto which the collector molecules are immobilised.

If the biosensor of the invention comprises several detectors, then preferably each detector is assigned to one field or one position of the matrix, even more preferably in that it is arranged below this field or position and the size of the measuring surface corresponds essentially to the field size.

In this respect, an embodiment in which the collector molecules are arranged in a support surface depression and on the base of the latter is preferred, whereby the base of the depression is lowered by at least 100 nm relative to the surface.

The invention concerns equally a method for detecting an analyte/collector complex by means of time-resolved luminescence using an optical biosensor in the form of a microchip, the biosensor comprising (a) a support with a surface onto which at least one type of collector molecule is immobilised, (b) at least one, preferably several detector(s) that can detect light passing through the surface, and (c) optionally at least one excitation source that can induce the emission of luminescence light, the method comprising steps (1) to (3), wherein in step (1) luminophores bound to the collector molecules and/or the analyte/collector complex are converted into an excited state for an excitation time T₁, in step (2) there is essentially no excitation for a die-away period T₂, and thereafter in step (3) for a period of time T₃ (measurement period) emitted luminescence light is detected by the at least one detector and is evaluated to detect the complex.

According to one embodiment, in step (3) various analyte/collector complexes can be detected in parallel, for example by parallel detection of luminescence light of different wavelengths. Equally, the method can comprise an additional step (4) in which, for a subsequent second measurement period T₄, luminescence light emitted at a wavelength different to that detected in step (3) is detected and is evaluated to analyse a second complex.

In all of the aforesaid cases the preferred method is one in which the excitation takes place only in step (1). The steps (1) to (3) or (1) to (4) can be performed several times.

In addition, the method of the invention described above can comprise a preceding step of bringing the collector molecules into contact with a sample that is assumed to contain a ligand for the collector molecules (=analyte), and optionally the washing of the biosensor(s).

According to one preferred embodiment, the analyte is labelled with a luminophore and detection does not take place until complex formation between the analyte and the collector molecules has taken place.

Preferably the luminophore is chosen from the group consisting of the rare earth metals or actinide metals, in particular europium, terbium and samarium; semiconductors of classes II-IV, III-V and IV, optionally doped, particularly CdSe, CdS or ZnS; and alkaline earth metal fluorides, in particular CaF, and mixtures thereof. The luminophore is most especially preferred in the form of nanocrystals, beads or a chelate.

The method can be performed specifically to detect a nucleic acid, nucleic acid analogues, a protein, peptide, haptene, antibody or fragment thereof, a sugar structure, a receptor or a ligand.

In every case it is preferable to use a biosensor as described above to carry out the method of the invention.

### BRIEF DESCRIPTION OF THE DRAWINGS

FIG. 1 shows diagrammatically a functional portion of a biosensor (1) according to the invention, manufactured by a CMOS process. The optical detector, e.g. a pn diode (2) is covered over with an insulator (e.g. field oxide) (4). In the region of the optical detector and/or detector field, the scratch protection (3) is either etched down sharp-edged or stepwise so that the collector molecules (e.g. DNA probes) (6) are arranged in a sunken area. The scratch-protection surfaces of the sensor chip, which play no part in the detection itself, can be modified by the application of, for example, noble metal or hydrophobic/hydrophilic materials (5).

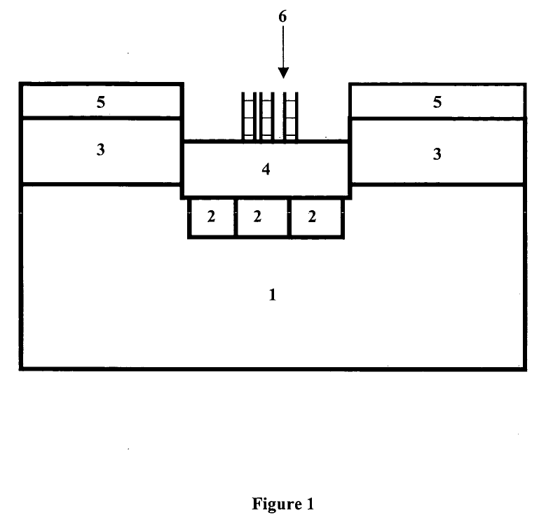

FIG. 2 shows that according to the invention it is also possible to provide, on the biosensor (1), detectors and/or detector fields that are, as indicated in the left-hand part of the illustration, not printed or coated with collector molecules such as, for example, DNA probes (6). The purpose of this is to compute out of the specific detection signal of the hybridised DNA (right-hand part of the illustration) the interference signals such as may be caused, for example, by the inherent fluorescence of the system components.

FIG. 3 shows that the biosensor (1) of the invention can be equipped with several photodiodes (2) per detection field, whereby the same type of collector molecules is immobilised above each detector of this field. This yields the possibility of multiple measurement for a given analyte/collector complex from which a statistical estimate of the specific measured signal can be derived. For example, this enables a differentiation between non-specific and specific signals.

FIG. 4 shows a detection field extended across several detectors (2), with the aid of which irregularities in the immobilising of the collector molecules (6) on the surfaces of the biosensor of the invention (1) can be balanced out.

### DETAILED DESCRIPTION OF THE INVENTION

Compared to the detection systems previously known, in which the luminescence-based detection of the presence of an analyte (ligand) in a sample that is to be analysed takes place via the specific bonding of the latter to a collector molecule directly or indirectly immobilised onto a solid phase and the subsequent measurement of the light intensity emitted by the collector/analyte complex takes place by the use of complex imaging optics such as CCD cameras, the present invention is based on the fact that these complex imaging optics are replaced by integrated means for a direct image recording process.

Thus, in concrete terms, the invention concerns an optical biosensor in the form of a microchip for detecting a collector/analyte complex by means of luminescence, the biosensor comprising (a) a support with a surface onto which at least one type of collector molecule is immobilised, (b) at least one detector that can detect light passing through the surface, and (c) optionally at least one excitation source that can induce the emission of luminescence light, wherein the surface is the detector measuring surface or a surface of a layer arranged above the detector without an interposed wavelength filter for light from the excitation source, i.e. the excitation wavelength. The invention concerns equally a method for detecting an analyte/collector complex by means of time-resolved luminescence using an optical biosensor in the form of a microchip, the biosensor comprising (a) a support with a surface onto which at least one type of collector molecule is immobilised, (b) at least one detector that can detect light passing through the surface, and (c) optionally at least one excitation source that can induce the emission of luminescence light (preferably by using the biosensor according to the invention and described above), the method comprising steps (1) to (3), wherein in step (1) luminophores bound to the collector molecules and/or the analyte/collector complex are converted into an excited state for an excitation time T₁, in step (2) there is essentially no excitation for a die-away period T₂, and thereafter in step (3) for a period of time T₃ emitted luminescence light is detected by the at least one detector and is evaluated to detect the complex.

According to the invention, the expression “luminescence” includes all emissions of light produced by an excitation source (and in the extended sense including the emission of ultraviolet and infrared radiation) from gaseous, liquid and solid substances that is caused not by high temperature but by prior absorption of energy and excitation. The substances that display luminescence are called luminophores. Although the present invention is explained in detail on some occasions by using the terms “fluorescence” and “fluorophore”, these terms merely describe preferred embodiments of the invention and thus do not constitute any limitation thereof.

As the person skilled in the art knows, luminescence can be produced by irradiation using a source of excitation with light (preferably short wavelength light as well as X-rays, photoluminescence), with electrons (cathodoluminescence), ions (ionoluminescence), sound waves (sonoluminescence) or with radioactive substances (radioluminescence), by electrical fields (electrochemiluminescence), by chemical reactions (chemiluminescence) or mechanical processes (triboluminescence). On the other hand thermoluminescence involves luminescence evoked or intensified by heating. All of these processes are subject to the general basic laws of quantum mechanics and cause excitation of the atoms and molecules which afterwards revert to the ground state with the emission of light which is detected in accordance with the invention. “Intrinsic fluorescence” (self-fluorescence) describes luminescence that can be excited in a substance or in an analyte without prior labelling with a luminophore.

Accordingly, the selection and if necessary differing construction of suitable excitation sources depends on which type(s) of luminescence generation is/are to be employed and/or on the luminophores used. Consequently the excitation source can be provided for example in the form of electrodes, light-emitting diodes, ultrasonic vibrations etc. The excitation source can preferably be integrated wholly or partially into the biosensor of the invention.

It has been known for a long time that the sensitivity and thus the lower limit of detection of relevant systems are restricted by a self-fluorescence inherent in the construction material components as well as by system-related light scattering. Attempts by the engineering industry to minimise the background noise caused by this and/or to obtain an optimised signal-to-noise ratio have led to, among other things, the technology of time-resolved or time-delayed (time-resolved) luminescence or fluorescence measurement, which has already been applied successfully in various areas of application.

The general principle of time-resolved luminescence and specifically fluorescence measurement is as follows: when a mixture of fluorescence compounds is excited by a short light pulse, for example from a laser or from a photoflash lamp, the excited molecules emit either a short-duration or a long-duration fluorescence.

Although both types of fluorescence decrease proceed exponentially, the short-lived fluorescence decays to a negligible value within a few nanoseconds. Provided that measurements are essentially absent during this short period of time after excitation has taken place, all of the background signals from the short-lived fluorescence as well as all of the radiation pulses caused by scattering are eliminated, with the result that the long-duration fluorescence signals can be measured with very high sensitivity.

Therefore the method of the invention comprises steps (1) to (3) in which, in step (1), luminophores bound to the collector molecules and/or the immobilised analyte/collector complex itself is converted into an excited state for an excitation time T₁, in step (2) essentially no excitation takes place for a die-away time T₂, and thereafter in step (3) emitted luminescence light is detected by the at least one detector for a period of time T₃ and is evaluated to detect the complex. According to the invention, preferably the measured values detected during T₁ and T₂ are not taken into account in the evaluation. More preferably no detection takes place during these times.

In this context, the expression “essentially not excited” (in translation: “essentially no excitation”) means that in contrast to the excitation time T₁, the excitation source is either completely switched off during the die-away time T₂, which is preferred according to the invention, or supplies to the system less than 10%, more preferably less than 5% and most preferably less than 2% of the energy per unit time (s) that is supplied during the excitation period. Most preferably the excitation source is not activated, i.e. does not supply any energy to the system, during the die-away time T₂ and during the measuring period T₃.

As a result of the use of the time-resolved luminescence measurement, which had been regarded in the prior art as not applicable for biosensors in the form of microchips, it has now astonishingly become possible in an advantageous manner to do without the wavelength filter(s) interposed between the signal origin location (surface-bound luminophore or complex emitting inherent luminescence, respectively) and the detection location (measurement surface of the detector(s)). Accordingly, the whole of the emitted luminescence light can be utilised for the measurement and/or detection, with the result that the sensitivity of the biosensor can be increased compared to the corresponding sensors of the prior art. The close positioning of the signal origination point and the detector (preferably less than or equal to 10 μm) makes a further contribution to this.

According to the invention, either the inherent luminescence of complexes (e.g. of proteins containing tyrosine) or the luminescence of luminophores previously introduced as markers into the analytes to be analysed can be used for this purpose. The latter is preferred. The luminophores especially suitable for this invention are those whose half-value time is distinctly more than 5 ns, so that these luminophores are still measurable after what is known as the fluorescence background (see above) has decayed away. The most preferred are luminophores whose half-value times are in the μs to ms range, particularly in the range between 100 μs and 2000 μs. Accordingly, the measurements in the context of the method of the invention are generally performed after the elapse of a period of time of approx. 5 ns after excitation has taken place. According to a preferred embodiment, the measurement window lies in the μs to ms range, whereby the range between 100 μs and 2000 μs is especially preferred.

Most organic luminophores show only a short half-value time in the excited state. This effect, known as fluorescence in the stricter sense of the word, is based on an electron being raised to a higher vibration energy level, the so-called excited singlet state, caused by the energy of the excitation source. This state has a stability of only a few ns (e.g. 2.6 ns for tryptophan). The excitation energy is then liberated as light as the electrons drop back from the excited singlet state into the ground state. As a rule the emission wavelength in this process is longer than that of the excitation source. The difference between the excitation wavelength and the emission wavelength is known as the Stokes Shift. On the other hand in the case of some luminophores a transition takes place from the excited singlet state into what is known as the triplet state. In this case the excited state is stabilised and the Stokes Shift is enlarged. As a rule this triplet state is on an energy level immediately below that of the excited singlet state. In the triplet state the electron is no longer spin-paired with the ground state of the electron. Thus the transition from the triplet state to the ground state involves a quantum-mechanically forbidden transition. This stabilises the lifetime of the excited state. This effect is called phosphoresence and has half-value times up to 10 ms.

The classical luminophores with a long half-value time include, for example, compounds of the rare earth metals (REMs) or actinide compounds, although because of their radioactivity the latter now play only a secondary role. In biology the RE metal ions are used mostly as chelate complexes, since the luminescence yield can be increased dramatically by choosing a suitable organic bonding partner. Such compounds are available commercially as what are known as “Microspheres” with a diameter of several hundred nm (e.g. FluoSpheres™Europium Luminescent Microspheres, Molecular Probes). Especially preferred RE metals are europium, terbium and samarium.

Other suitable luminophores are nanocrystals of semiconductors, whose special property in addition to their luminescence properties is a relatively small size (a few nm) and high stability (no photobleaching). The half-value time here, depending on the semiconductor material and doping chosen, can be adjusted in a wide range from several hundred ns to the millisecond region. The person skilled in the art can easily coat appropriate nanoparticles with, for example, silanes and afterwards couple them to organic molecules, e.g. nucleic acids or antibodies. Suitable semiconductors include semiconductors of classes II-VI (MgS, MgSe, MgTe, CaS, CaSe, CaTe, SrS, SrSe, SrTe, BaS, BaSe, BaTe, ZnSe, ZnTe, CdS, CdSe, CdTe, HgS, HgSe, HgTe), Ill-V (GaAs, InGaAs, InP, InAs), and IV (Ge, Si). Semiconductor crystals of this nature have half-value times in the region of approx. 200 ns or more. Nanocrystals of class II-VI are available as what are known as “Quantum Dots®” (Quantum Dot Corp., California, USA). In each case the absorption spectrum of nanocrystals within a class is identical, but the respective emission spectra differ as a function of the given particle size, so that when using filter optics several parallel labellings can be measured using a single excitation wavelength. Table 1 shows the properties of some nanocrystals.

TABLE 1

| Nanocrystals | Size | Extinction | Emission |
|---|---|---|---|
| CdSe—CdS | 2.4 nm | 350-450 nm | 533 nm |
| CdSe—CdS | 4.6 nm | 350-450 nm | 630 nm |
| Mn²⁺-doped ZnS | 1.5-3 nm | 230-320 nm | 550-650 nm |

Other substances with marked luminescence that are suitable in the context of the present invention are crystals of cadmium selenide, cadmium sulphide or zinc sulphide doped with manganese, copper or silver. The self-luminescence of these substances is caused by defects in the crystal lattice. Different emission spectra can be produced by selecting different metal ions for doping (Ag, Cu, Mn). As the said substances are insoluble in water, it is preferable to use them according to the invention in the form of what are known as microparticles. The properties of some representatives of this group of substances are illustrated by Table 1 above, taking Mn²⁺-doped ZnS as an example.

Other luminophores that are suitable according to the invention are the alkaline earth halides with lattice defects that can be produced, for example, by doping (foreign ions) or radioactive irradiation. For example calcium fluoride (CaF) particles show marked luminescence when doped appropriately, e.g. with europium. In the case of CaF, for example, thermoluminescence can also be produced with radioactively generated lattice defects, whereby temperatures as low as about 40° C. are sufficient to trigger luminescence.

The fluorescing rare earth metal compounds or chelates preferred for use in the context of the method of the invention, e.g. particularly the europium chelates, were chosen because of certain advantages compared to conventional fluorophores as markers (labels) for time-resolved fluorimetry. The fluorescing europium chelates have large “Stokes Shifts” (approx. 290 nm) without any overlap between the excitation and emission spectra and are characterised by a very narrow (10 nm bandwidth) emission spectrum at approx. 615 nm. Moreover, their long fluorescence half-value times (600-1000 μs for Eu³⁺ compared to 5-20 ns for conventional fluorophores) allow the use of time-resolved fluorescence measurements in the micro to millisecond region, as a result of which the aforementioned background signals can be reduced.

TABLE 2

| Metal ion (half-life) | State | Extinction | Emission |
|---|---|---|---|
| Eu³⁺ (600 μs) | Microspheres | 340-370 nm | 610 nm |
| Tb³⁺ (approx. 1 ms) | NTA complex | 270 nm | 545 nm |
| Pt²⁺ (>100 μs) | Microspheres | 390 nm | 650 nm |

NTA: 2-(Trifluoroacetyl)-naphthalene, Molecular Probes

The use of europium chelates as labels in the context of time-resolved fluorimetry has already been known for a long time from immunological assays as well as from Southern and Western Blot applications. The appropriate labelling of the biomolecule (analyte) that may possibly be present in a sample for analysis can take place based on established protocols either with Eu³⁺ or an Eu³⁺-chelating agent (see, for example, E. P. Diamandis and T. K. Christopoulos, “Europium chelate labels in time-resolved fluorescence immunoassays and DNA hybridization assays”, Anal. Chim. 62: 1149A-1157A (1990)).

According to a preferred embodiment of the method of the invention, alternatively the analyte can be biotinylated and detection can take place using Eu³⁺ or an Eu³⁺-chelating agent coupled to streptavidin. The technique especially preferred in this respect is the use of what are known as “Beads” that are charged with the appropriately chosen rare earth metal compounds, thus ensuring a very high density of luminescence-emitting molecules. It is known from empirical experiments that the detection limit of fluorimetry systems optimised in this way is approx. 1 to 5 pg of protein or DNA.

The biosensor according to the invention comprises a support (substrate) with a surface that is preferably flat or equipped with suitable depressions (wells) onto which at least one type of collector molecule, preferably several types of collector molecules is/are immobilised. The immobilisation preferably takes place via direct or indirect (for example by using ‘Spacers’) covalent bonding to the surface. Appropriate coupling techniques are known to the person skilled in the art. Preferably the collector molecules are selected from the group consisting of single or double-stranded nucleic acids, nucleic acid analogues, haptenes, proteins, peptides, antibodies or their fragments, sugar structures, receptors or ligands. DNA is especially preferred.

Basically the support for the biosensor of the invention is made from any suitable material that is sufficiently transparent at least in the region in which the collector molecules are immobilised. Suitable materials include rigid and flexible materials, for example plastics films, polymers, glass, rigid plastics, silicon, silicon nitride, silicon oxide, aluminium, aluminium oxide and other materials known from semiconductor technology, and especially direct semi-conductors. The latter are usually preferred. The support usually has a planar configuration, for example in the form of a microchip, and can have dimensions of up to 5 cm, preferably 1 to 5 cm wide, up to 10 cm, preferably 2 to 10 cm long and up to 0.5 cm, preferably 0.1 to 0.5 cm thick. The expression “microchip” as used herein does not necessarily imply the properties of a microchip as known from electronics. Basically the expression relates first of all to the planar method of construction together with the dimensions, which differ markedly from conventional optics. Another essential feature is the provision of a (preferably planar) surface onto which the collector molecules can be immobilised. However, the use of a “microchip” in the conventional sense is preferred. A microchip of this kind is usually a monolithic combination, i.e. fabricated from a single piece, of various semiconductor materials such as, for example, silicon, silicon dioxide, silicon nitride, aluminium, aluminium oxide etc.

For example the measuring device known from EP-A-0 881 490 for measuring certain physiological and morphological parameters of at least one living cell to be studied can be used, after appropriate modification, to carry out the method of the invention. The device described already has numerous sensors and/or detectors that are an integral part of a support device onto which the material to be studied is immobilised.

According to a preferred embodiment, the support can consist essentially of a semiconductor material with an optical detector layer, preferably comprising several detectors, built into it, whereby it is preferable to incorporate photodiodes as detectors. This layer can be incorporated monolithically into the support (microchip in the narrower electronic sense). Alternatively it can be attached by adhesive to the underside of the support, the collector molecules being immobilised onto the upper surface of the latter.

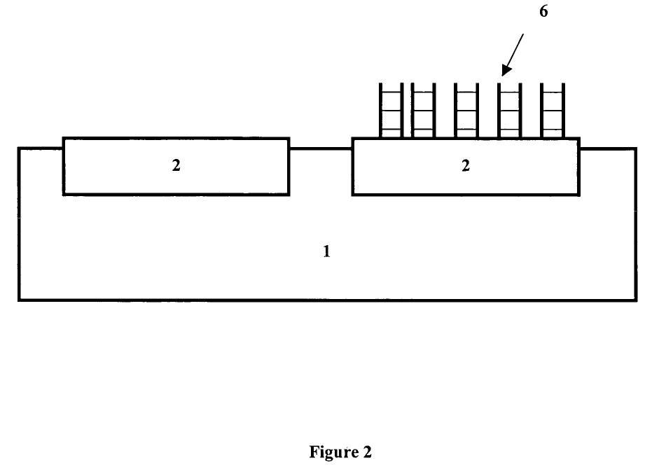

In a particularly preferred embodiment, the signal processing takes place at least partly within the biosensor. According to one aspect of the present invention, the time-resolved fluorescence can, for example, be evaluated directly on the microchip by using analogue circuits, by recording a value, e.g. for each nanosecond after switching off the excitation source, and then comparing these (recorded values) with a reference value of a measurement carried out previously and which was also stored on the microchip. Furthermore, this procedure enables non-specific interfering signals such as, for example, the self-fluorescence of system components that may possibly be present, to be computed out (see also FIG. 2). Assuming that in the meantime it is possible to resolve even into the GHz region (<1 ns), it will be possible to distinguish the self-fluorescence from the artificial fluorescence.

Provided the support surface is designed as a microarray arrangement in which a plurality of detection fields are to be evaluated, the detection of the luminescence signals can take place sequentially by, for example, successively exciting and detecting entire lines or columns of the surface or parts thereof (multiplex application).

For example the electronic output signals from the detectors, after analogue-digital conversion, can be carried to an external evaluation device by means of suitable circuitry. In addition to the photodiode (pn, p-i-n, avalanche), it is also possible to consider CCD sensors or photoconductors as suitable optical detectors or sensors according to the invention, preferably incorporated monolithically into the semiconductor substrate of the biosensor in the form of a linear or array arrangement (=pattern). Photodiodes can be used advantageously in the context of time-resolved luminescence measurement because they have a small detection surface area or measuring surface area compared to photomultipliers.

According to one particularly preferred aspect of the present invention, the excitation source is an integral constituent of the biosensor (for example in the form of electrodes) and most preferably is provided by the detector itself. The choice of a pn diode made of direct semi-conductor material enables the following: in the first case activation means applying a voltage as a result of which a light signal is emitted (pn diode used as an LED) lying in a particular emission wavelength band depending on the type and nature of the pn diode and causing the excitation of an analyte bound in the proximity of this pn diode. After deactivating the pn diode (pn diode being used as a photodiode) and after the elapse of a certain die-away time, it is then activated once again in order to carry out the required measurement(s).

As a result of the fact that the excitation radiation in the embodiment described above is coupled in via the same components with which the luminescence radiation is also collected, it is possible to achieve a situation in which a very small region of the sensor surface or of the detection field is selectively irradiated and luminescence radiation emanating from this region is evaluated. As a result of this procedure, it is possible to image the detection field under study very precisely and to avoid interference with the measurement by luminescence from outside the region being studied.

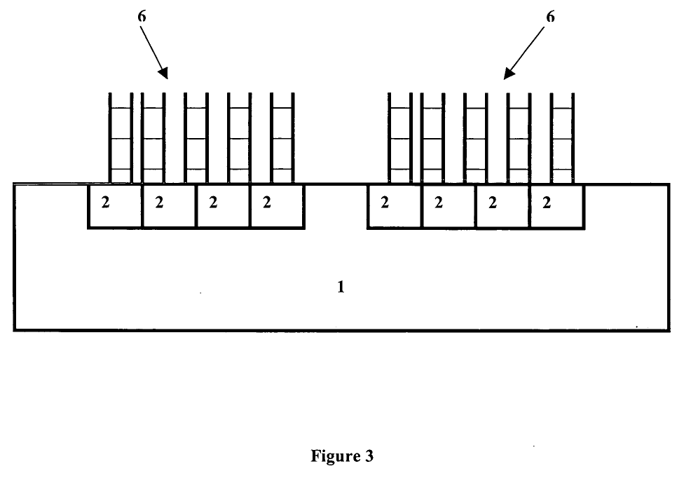

Of course the detectors can, in addition, be arranged in groups, whereby it is possible to create individual detection fields whose input signals ensure a result that is more reliable than would be possible with single occupation per detection field (see FIG. 3). By multiple occupation per detection field it is also possible to ensure centring of the analyte binding event by measurement technology, which can contribute to a distinct increase in sensitivity during the signal processing.

The manufacture of a biosensor according to the invention can take place using the CMOS (complementary metal oxide semiconductor) process which is itself known, and for this reason all of the circuit libraries for integrating the signal conditioning and evaluation are available without modification and can be implemented in the context of the present invention. A comprehensive presentation can be found, for example, in WO 99/27 140. Examples of other manufacturing processes that are also suitable according to the invention are NMOS processes or bipolar processes. Another possibility, which is attractive particularly from the point of view of costs, is to manufacture a biosensor of the invention based on organic semiconductors (see, for example, EP-A-1 085 319).

According to a further preferred embodiment, the individual detection fields are separated from one another in such a way that essentially no light emission from one field can be received by the detector(s) of another field. Thus the individual detector fields can be arranged in depressions (wells) of the kind that are known from the usual microtitration plates. According to the invention, trough-shaped depressions and depressions with bottoms whose sidewalls are arranged essentially perpendicular relative to the surface of the sensor chip are preferred. The respective dimensions of such a depression can be chosen freely by the person skilled in the art based on a knowledge of the area of application, provided that the luminophore(s) of the expected analyte/collector complex are situated inside the depression, preferably on its base, and essentially no emitted light can penetrate into neighbouring depressions.

In one especially preferred depression, its base is recessed by at least 100 nm, preferably 100 nm to 10 μm and more preferably 100 to 5000 nm into the surface of the biosensor of the invention. Alternatively the same effect can be achieved by arranging, on the essentially planar surface, means of separation pointing upwards, whose dimensions can easily be chosen by the person skilled in the art from a knowledge of the required area of application and the geometrical dimension of an anticipated collector/analyte complex. For example the installation of appropriate suitable means of separation can take place by anodic bonding or by what is known as the Flip-Chip process.

In a preferred embodiment, channels are applied to the biosensor in the form of a microchip. For example the channels can supply rows of detection fields onto which the arrays of collector molecules are bound. For example this allows calibration measurements to be carried out. In a further preferred embodiment a parallel measurement can be performed on identical arrays, e.g. on parallel samples, in order to reduce the costs per analysis dramatically in this way. For this purpose the microchip is sub-divided by microchannels, for example into 8 identical compartments.

It is obvious to the person skilled in the art that the choice of the support material, the surface and the detector(s) depends on the luminophore emission wavelength that is to be detected.

Basically it must be said that the detector has differing sensitivities with regard to wavelength depending on the choice of material (e.g. silicon or germanium) because of what is known as the “semiconductor band gap”. Therefore, in the preferred case of the use of a silicon photodiode, a sensitivity range extending from the infrared into the ultraviolet wavelength spectrum is created, the sensitivity being greatest between these regions.

Furthermore, according to a preferred embodiment, the biosensor of the invention can comprise one or more additional elements from the group consisting of a control unit, at least one amplifier, one or more signal transformers, one or more storage/memory units, one or more filters, an optics system, light guides (optical fibres) and one or more protective layers; this subject to the proviso that no wavelength filter for light from the excitation source or the latter's wavelength is arranged or interposed between the detector(s) and the surface of the support onto which the collector molecules are immobilised. Most preferred is the embodiment in which the collector molecules are immobilised on the measuring surface of the detector (for example the uppermost layer of a pn diode).

If a monolithically integratable semiconductor material is used as the support and surface for the collector molecules and to form the detectors, it is also possible to construct a monolithically integrated circuit on the same substrate, as a result of which pre-processing of the electronic detector output signals can take place in the immediate vicinity of the subject of the examination (collector/analyte complex). Thus this preferred embodiment of the present invention involves an “intelligent” biosensor that performs considerably more than purely passive sensors. For example the output signals from the electro-optical detectors can be preprocessed by a co-integrated circuit in such a way as to enable them to be relayed via output circuits and connector contacts to the outside world and processed, i.e. evaluated there in a relatively problem-free way. Furthermore, the pre-processing can consist of digitising the analogue detector signals and converting them into a suitable data stream.

Moreover, because of the short signal paths, the signal-noise ratio can be improved very greatly by the proximity of the detector to the signal-processing location that is implemented in the biosensor according to the invention. Furthermore, it is also possible for there to be additional processing steps with which, for example, the amount of data can be reduced or which assist the external processing and presentation. This enables the remaining evaluation of the optical signals and their presentation to be carried out by a Personal Computer (PC).

Furthermore, the biosensor of the invention can be constructed in such a way that the data, preferably compacted and/or processed, can be transmitted to correspondingly equipped reception stations via an infra-red or wireless link.

The control of the associated devices on the substrate can take place via control signals from a control device, which is preferably also entirely or partially constructed on the substrate, or one which is connected externally.

The possibility of evaluating the optical/electrical signals in the context of the method of the invention via an ordinary commercially-available computer has the added advantage that extensive automation of the data evaluation and storage via suitable programs is possible, with the result that, in the context of the data analysis, there are no restrictions of any kind compared to data generated using conventional external imaging optics as a result of the use of the biosensor of the invention.

The direct recording of the luminescences from the biosensor of the invention is achieved in that the collector molecules necessary for a specific detection are situated, directly or via a usual distance holder (spacer) and/or coupling matrix, on a surface that is either the measuring surface of the detector or the surface of a layer arranged immediately above this measuring surface without any interposed wavelength filter. This arrangement helps to reduce the distance between the place where the signal originates (emission of luminescence light) and the detection location, thus maximising the yield of luminescence light.

According to a preferred embodiment, the optical detector is provided in the form of at least one photodiode, whereby the presence of a plurality of these photodiodes for, inter alia, the parallel and/or sequential detection of several different analytes (ligands) is especially preferred. However, even when using only one type of luminophore, this multiple arrangement offers the advantage that, as a result of several detectors per detection field, it is possible to record profiles with whose aid the site-specific assignment of a binding event of collector molecule and analyte can be improved by way of centring. In the context of these embodiments, which are aimed specifically at the micro-array arrangements that are already known as such, the individual photodiodes can be grouped together advantageously into defined groups or measuring fields, as a result of which the sensitivity of the subsequent luminescence measurement and the reproducibility and reliability of the measured data obtained thereby can be increased significantly.

According to a preferred embodiment the surface, optionally exposed (otherwise the biosensor is covered, e.g. by a protective layer), of each photodiode (as detector and optionally simultaneously as excitation source) consists of SiO₂ or Si₃N₄. Furthermore, certain process parameters of the collector/analyte bonding and the detection can be influenced beneficially by the choice of the surface material for the biosensor in the form of a microchip. For example Si₃N₄ can be applied at some places while SiO₂ (or e.g. Al₂O₃) or a noble metal is applied at another, as a result of which preferred regions for the biomolecules or spacers with, for example, more hydrophobic or more hydrophilic properties can be provided on the sensor or even in individual detection fields in order to promote or suppress in a localised manner the attachment of, for example, DNA as a collector molecule. In addition, according to the invention, the attachment of drivable noble metal electrodes can enable the creation of preferred biosensors in which, for example, hybridisation events can be accelerated or a fluorescence originating from electrically excitable luminophores (electrochemiluminescence) can be triggered by the application of different potentials, optionally for each detection field.

The excitation source which, for example, can be provided in the form of one or more white light lamps, LEDs (light-emitting diodes), (semiconductor) lasers or UV tubes, as well as by piezo-elements (ultrasonics) or by gases and/or liquids emitting light energy (chemical excitation) or by electrodes, should be sufficiently powerful and preferably repeatable at high frequency. The latter property exists if the light source can be both activated and extinguished for short periods of time. If an optical excitation source is used, this should be capable of being switched off in such a way that essentially no further photons impinge on the detectors after switching off (for example as a result of after-glow), i.e. no energy in the aforesaid sense is supplied to the system. If necessary this can be ensured by using mechanical closure apertures (English: “shutters”) as well as by choosing LEDs or lasers as the optical excitation source.

It is preferable for the excitation source to be coupled optically and mechanically to the biosensor and the detectors in such a way as to generate a radiation field in the direction of the latter, whereby the spacing distance between the excitation source and the signal origin plane, i.e. the surface onto which the collector molecules are immobilised, is as small as possible.

However, the spacing distance must be sufficiently large that the reactions between ligand/analyte and collector molecule that are necessary for the use for which it is intended are not impaired. In this respect it may be appropriate for the excitation source—corresponding to the plurality of detection fields provided on the support—to consist of a plurality of point-sized radiation sources that can be activated individually or in groups, for example by means of a control device. The simultaneous use of a pn diode as the excitation source (LED) and detector (photodiode) is especially preferred here. The irradiation can take place directly, i.e. without any interposed optics, provided the light beam emitted by the excitation source is already highly focussed, in order to ensure vary small detection fields, particularly in the context of the use of what are known as micro-arrays. Alternatively, however, the radiation path from the excitation source can be focussed by using suitable lenses insofar as this is advisable because of, for example, the very dense population of collector molecules on the sensor surface. It is clear to the person skilled in the art that this provides a further means to reduce non-specific interference signals, e.g. self-fluorescence.

The arrangement of the point-sized radiation sources, consisting for example of focussed optical fibres or miniaturised LEDs (Light Emitting Diodes) or implemented in some other way, is advantageously in the form of lines or fields and thus functionally adapted to the arrangement of the collector molecules on the sensor surface. For use in the context of analyses using various RE (rare earth) metal chelates, it may be advantageous for the excitation sources to be fully tunable or for excitation sources for different wavelengths to be present. In addition it may be advantageous for particular intended purposes for the excitation source to be frequency modulated. In this connection, intensity-modulated excitation light is used, whereby the modulation takes place at several MHz when measuring half-life times in the nanosecond range. Therefore the method known to the person skilled in the art by the name FLIM (English: “Fluorescence Lifetime Imaging Microscopy”) is included in a further preferred embodiment according to the invention. The above statements mean that different or fully tunable detectors can be present on the detector side to record the light energy emitted by the collector/analyte complex. Where this involves photodiodes, wavelength-specific photoelements are used for the biosensor of the invention.

Biosensors with conventional photodiodes equipped with superimposed, applied, vapour-deposited or integrated wavelength filters are also suitable for carrying out the method of the invention. Thus in contrast to silicon dioxide, for example, it is known that silicon nitride is opaque to UV light and that polysilicon absorbs UV radiation. Therefore nitride or polysilicon can be deposited on the gate oxide layer in the context of the usual CMOS process, resulting in the creation of the corresponding filters on the photodiode. Thus NADH (nicotinamide adenine dinucleotide), for example, has an excitation wavelength of 350 nm and an emission wavelength of 450 nm. Therefore the sensitivity can be increased by applying a filter that filters out 350 nm. This effect can be used for the method of the invention to enable differential detection, for example when using two different luminophores in parallel, of which, for example, only one emits light in the UV region, since the detectors provided for this purpose are or are not of UV-sensitive construction. In addition this effect offers an opportunity where necessary to exclude from the measurement process the interfering self-fluorescences of materials with a known emission wavelength that may be present by providing appropriate filters.

An example of this is the parallel use of europium chelates (emission at approx. 620 nm) and zinc sulphide doped with copper (emission at approx. 525 nm), which, as a result of emission wavelength ranges that are sufficiently different from one another, enable two-colour detection, e.g. within one region of a detection field, by for example equipping half of the detectors of a detection field with a low-pass filter and the other half of the detectors of the same field with a high-pass filter. However, the most preferable way to improve the sensitivity is to omit the use of wavelength filters.

Additionally or alternatively, different luminophores can be used in parallel provided that their physical and/or optical properties are sufficiently dissimilar. For example the differing excitation wavelengths of two luminophores A and B that are to be used and/or their differing half-value times are utilised. This can take place, for example, by providing two differently doped nanocrystals. In the latter case of differing half-value times the emissions can be recorded in two consecutive measuring times T₃ and T₄.

The detection or determination and optionally quantification of the complexes for a specific collector/analyte necessitates the fixation and preferably immobilisation of at least one type, preferably several types of collector molecules on the surface of the biosensor support. According to a preferred embodiment, this immobilisation can take place using a coupling substance that is deposited as a layer on the surface. For this purpose, typically the biosensor surfaces made of metal or metalloid oxides, e.g. aluminium oxide, quartz glass or glass are dipped into a solution of bifunctional molecules (known as “linkers”) having for example a halogen-silane (e.g. chlorosilane) or alkoxysilane group to couple to the support surface, with the result that a self-organising monolayer (SAM) is formed, via which the covalent bonding between the sensor surface and receptor is created. For example the coating can be performed with glycidyl triethoxy silane, which can take place for example by immersion in a solution of 1% silane in toluene, slow withdrawal and immobilisation by “baking” at 120° C. A coating produced in this way generally has a thickness of a few Angstroms. The coupling between the linker and collector molecule(s) takes place via a suitable additional functional group, for example an amino or epoxy group. Suitable bifunctional linkers for coupling numerous different receptor molecules, including especially those of biological origin, to a plurality of support surfaces are well known to the person skilled in the art.

Insofar as the biomolecules to be detected involve nucleic acids, suitable DNA probes as collector molecules can afterwards be applied and immobilised using currently available printing equipments.

Hybridisations with, for example, biotinised DNA, can now be performed on biosensors manufactured in this way by the use of established methods. For example these can be generated by means of the PCR (Polymerase Chain Reaction) and the incorporation of biotin-dUTP. During hybridisation, the biotinised DNA bonds to the complementary strand (where present) immobilised onto the biosensor in the respective detection field. Positive (successful) hybridisation events can then be demonstrated by adding conjugates of streptavidin/avidin and luminophore. According to the invention, the following are especially suitable as luminophore conjugates: europium, terbium and samarium chelates, and microspheres (“beads”) loaded with Eu, Sm and/or Tb chelates via avidin/streptavidin. Especially suitable in this respect are luminescent microspheres such as, for example, FluoSpheres Europium (Molecular Probes F-20883) because they are able to immobilise a large number of fluorochromes with a single bonding event. Also suitable according to the invention are nanocrystals of the kind offered by, for example, the Quantum Dot Corp. under the name “Quantum-Dots ®”. After washing to remove unbonded labelled analytes and/or free-floating luminescence dyestuffs, measurement of the bonding takes place by suitable excitation and measurement of the time-resolved fluorescence with the excitation light source switched off.

According to the invention, the excitation period (excitation time) is described as T₁, the period of time between excitation and measurement (die-away time) as T₂ and the measurement time period (measurement duration) as T₃, and if necessary a second measurement period as T₄. Preferably the time T₁ is 1 nanosecond to 2 milliseconds, the time T₂ 1 nanosecond to 500 microseconds, preferably 1 to 5 ns, and the time T₃ 5 nanoseconds to 10 milliseconds, preferably 5 ns to 2 ms.

The method of the invention can comprise an additional prefaced step of bring the collector molecules into contact with a sample that is assumed to contain a ligand for the collector molecules and if necessary washing the biosensor. Preferably the analyte is labelled with a luminophore and detection does not take place until complex formation between the analyte and the collector has taken place.

The signals from the detector or detectors are recorded by a recording unit. A recording unit has a very fast converter to transform analogue detector signals into digital values that are stored. Evaluation of the digital values is carried out preferably in real time, however it can also take place after a time delay. An ordinary microprocessor can be used to evaluate the digital values. This evaluation takes place only during the measuring period T₃ and if necessary T₄.

In the event that the luminescence signal is too weak for unambiguous detection, an enhancement of the detection sensitivity can be achieved in the context of a preferred embodiment of the detection by integrating several individual measurements. In doing this, an identical measurement takes place several times (steps (1) to (3) or (1) to (4) are traversed several times) and the results of measurement are summated. This can take place either directly on the sensor chip or via suitable software after the measurement.

For example, an individual measurement comprising steps (1) to (3) has the following appearance: during the excitation time T₁ the photodiode as a detector is in an insensitive mode relative to the excitation state. The excitation source is active during this period of time. During the period of time T₂, both the excitation source and the photodiode are inactive. During this time the background luminescence is able to die away. During the period of time T₃ the photodiode is active and detects between one and several incident photons from the luminophore. The detection process can be repeated by resetting the photodiode into the inactive mode. The respective time intervals can be chosen as, for example, 2 ms (T₁), 5 ns (T₂) and 2 ms (T₃). Given an appropriate signal strength, the time interval T₃ can also be markedly shorter than the half-value time of the excited state of the luminophore(s) used.

According to an especially preferred embodiment of the repetitive excitation, the detector values obtained in the time interval T₃ , optionally after digitising and further electronic processing, are stored in memory cells assigned to the individual time intervals. For example a storage memory of this kind has 100 or more memory cells allocated to successive time intervals. Such a time interval preferably lies in the range from 1 to 100 nanoseconds.

As a special preference, the signal obtained from the detector can also be analysed with regard to the signal intensity and a determination made as to the number of individual molecules (number of collector/analyte complexes) from which the signal originated, thus enabling not only qualitative but also a quantitative analysis. The multiple of the unit value corresponding to the number of luminophores is now stored in the memory cell.

The memory storage process described above takes place anew for each individual measurement, a summation being performed if repeated excitation is required, i.e. steps (1) to (3) are traversed several times. In this process the unit value, or if necessary a multiple thereof, stored in a particular memory cell after a measurement is added to the value already present in the cell. The summated curve obtained in this way with the measurements for a particular detection field can be evaluated to ascertain which and/or how many luminescent analytes are bound in the detection field. In principle those evaluation methods that are also used for signal curves obtained with a plurality of different analytes can be applied to the summation curves. An absolute recording of the luminescence events with a precision of a few picoseconds enables a global analysis of the photon statistics. Characteristic accumulations or pauses in the global photon distribution can be recognised and determined. This enables measurement of the triplet duration of a system and the determination of reaction kinetics. Equally it is possible in this way to measure diffusion times through the detection volume, which enable conclusions regarding the size of the analyte molecule. A total photon collection efficiency of 5 to 10% relative to the number of photons in the input radiation can be achieved with such a system. This results from a luminophore absorption efficiency of about 80%, an emission probability of about 90% and a detector sensitivity of up to 70%.

In this application, the control unit is preferably designed to activate the excitation source for a time interval T₁ and, after the elapse of a time interval T₂, to activate the detector for a time interval T₃. A control unit of this kind can enable a time-resolved luminescence measurement. The purpose of the time interval T₁ for which the excitation source is activated is to transfer the luminophores immobilised on the analytes and thus bound in the complex into an excited state from which they undergo transition into a lower-energy state accompanied by the emission of luminescence light. The purpose of the die-away time T₂ is to exclude from the measurement any spontaneous luminescence of the sample and/or of the support material that does not emanate from the group of molecules that are to be detected.

The detector(s) is/are activated at least during the measurement period T₃ (and optionally T₄) and receive(s) luminescence radiation from the collector/analyte complex. The chosen time T₃ is preferably between 5 ns and 2 ms. During this time T₃ the detector signals are acquired by a recording unit with regard to their signal height and timing and are afterwards evaluated. Where the measurement is performed on individual or at least very few molecules, what is obtained in the time interval T₃ is not a classical luminescence decay curve but for example in the case of a single molecule it is a signal peak characterising the moment in time or time interval in which the individual molecule emits radiation. As a result of carrying out the measurement repeatedly, it is possible for there to be a statistical evaluation from which the luminescence lifetime can be ascertained.

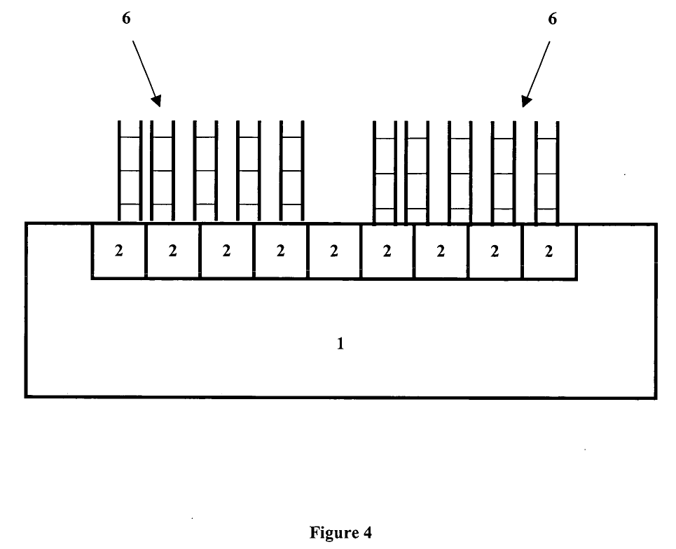

In an especially preferred embodiment it is possible to make provision for the signals from the as yet unhybridised DNA as collector molecule and/or from detectors onto which no collector molecule has been immobilised (background luminescence) and/or from unlabelled collector/analyte complexes, i.e. those not displaying any luminophore (for example hybridised DNA without a luminophore) to be stored in memory as reference or control values in order to have the ability to compute the recorded “interference signals” out of the detection signal during the actual detection event with the luminophore (see FIG. 4).

It is possible to summate the intensities obtained in a defined time interval within T₃ in the context of a statistical evaluation.

The way to use the method is clear to the person skilled in the art. As a supplement, attention is drawn to the statements in WO 98/09154.

The present invention will be illustrated in detail below using Examples and by reference to the attached Drawings.

### EXAMPLES

Manufacture of a Biosensor According to the Invention in the Form of a Microchip

The sensor is fabricated using 6″ (inch) wafers with a 0.5 μm CMOS process. Each pn photodiode is arranged in an n-trough on p-substrate. After the field oxidation, the definition of the p-regions of the photodiode and the application of the 10 nm thick gate oxide layer take place. This is followed by the superimposition and structuring of a silicon dioxide layer. Finally the other usual CMOS steps are performed, such as for example the application of a wiring layer and surface passivation (scratch protection).

Coating the CMOS Biosensor

The CMOS sensor manufactured as above is coated with the silane by dipping it into a solution of 1% GOPS (glycidyloxypropyl triethoxysilane) and 0.1% triethylamine in toluene for a period of approx. 2 hours. Afterwards the microchip is removed from the solution and after dripping dry for a short time it is fixed in a drying cabinet at 120° C. for a period of about 2 hours. The microchip coated in this way can be stored in the absence of moisture until bioconjugation.

Bioconjugation with Oligonucleotide Probes

Using conventional techniques, the microchip coated as above is printed by a non-contact process with 5′-amino-modified oligonucleotide probes. For this purpose the oligonucleotide probes are prepared in solution at a concentration of 5 μM in PBS buffer. After printing, the coupling reaction is continued at 50° C. in a humid chamber. Next the microchips are rinsed with distilled water and then dried by washing with methanol. Any remaining solvent residues are afterwards removed by evaporation under a fume hood.

Obtaining the Samples

Fragments of the haemochromatose gene are amplified from human DNA isolates by using PCR (=the Polymerase Chain Reaction). Suitable primer sequences are used in the amplification, for example as described in U.S. Pat. No. 5,712,098.

The reaction mixture contains the following standard reagents (primer: 0.5 μM, dATP, dCTP, dGTP: 0.1 mM, dTTP 0.08 mM, PCR buffer, MgCl₂: 4 mM, HotStarTaq (Perkin Elmer) 2 units/50 μl) plus the addition of Biotin-11-dUTP (0.06 mM). During the PCR reaction (35 cycles, 5 min. 95° C., 30 sec. 95° C., 30 sec. 60° C., 30 sec. 72° C., 7 min. 72° C.) the biotin-dUTP is incorporated into the newly-synthesising DNA. Afterwards single-stranded DNA is generated by adding T7 Gen6-exonuclease (100 units/50 μl of PCR mix) and heating the mix (30 minutes at 37° C., 10 min. at 85° C).

Hybridising

The above reaction batch is hybridised onto the microchip in a buffer of 5×SSPE, 0.1% SDS (12 μl) under a microscope cover glass for a period of 2 hours at 50° C. in a humidity chamber. Next it is rinsed with 2×SSPE 0.1% SDS and the microchip is cleaned by washing in water.

Labelling

A labelling solution consisting of 5% BSA, 0.2% Tween 20 and 4×SSC buffer and in which 0.001% solid microspheres (Europium Luminescence Microspheres, Neutravidin-coated 0.04 μM, Molecular Probes F 20883) are suspended is poured onto the microchip to perform the labelling. The reaction is carried out for a period of 30 minutes with agitation by an eccentric tumbler mixer. Next any non-bonded microspheres that may be present are removed from the mix by washing 2×SSC, 0.1% SDS.

Preparing Anti-Digoxigenin-IgG-Coated Microspheres

0.04 μM of Fluospheres Platinum Luminescent Microspheres (F20886) are modified with a monoclonal anti-digoxigenin-IgG antibody (goat) according to the instructions from the manufacturer (Molecular Probes) of the microspheres. The coated microspheres are afterwards dialysed against PBS in a dialysis sleeve with an exclusion size of 300 kD with five changes of buffer.

Two-Colour Detection on the Sensor Chip

Two PCR products are prepared as described above, whereby in one of the products the biotin-11-dUTP is replaced by an equimolar amount of digoxigenin-dUTP. Both reaction batches are treated in the same way and then hybridised in a 1:1 mixture on the microchip. Labelling takes place with a 1:1 mixture of the above solid microspheres (Europium Luminescence Microspheres, Neutravidin-coated 0.04 μM, Molecular Probes F 20883) and the antibody-modified spheres in the labelling buffer described there. Excitation of the platinum spheres takes place at 400 nm (light source: xenon lamp and monochromator) while that of the europium spheres is carried out at 370 nm (xenon lamp and monochromator). Illumination of the microchip takes place via an optical fibre and the luminescence spectra of the dye-stuffs are recorded separately. Alternatively illumination is with UV-LEDs (without a filter) and the luminescences of both dyestuffs are recorded and afterwards evaluated via the profile of the luminescence decay kinetics.

1-29. (cancelled)

30. Optical biosensor in the form of a microchip for detecting a collector/analyte complex by means of luminescent, comprising
(a) a support with a surface onto which at least one type of collector molecule is immobilised, and
(b) at least one detector that can detect light passing through the surface;
wherein the surface is one of (i) a measuring surface of the detector, and (ii) a surface of a layer arranged above the detector, without any interposed wavelength filter for light from an excitation source.

31. Optical biosensor in the form of a microchip for detecting a collector/analyte complex by means of luminescence, comprising
(a) a support with a surface onto which at least one type of collector molecule is immobilised,
(b) at least one detector that can detect light passing through the surface, and
(c) at least one excitation source that can induce the emission of luminescent light;
wherein the surface is one of (i) a measuring surface of the detector, and (ii) a surface of a layer arranged above the detector, without any interposed wavelength filter for light from the excitation source.

32. Biosensor according to claim 31, wherein said excitation source can induce a luminophore to emit luminescent light.

33. Biosensor according to claim 30, wherein said surface is a surface is disposed above a measuring surface of the detector by not greater than 10 μm.

34. Biosensor according to claim 30, wherein said collector molecules are covalently bonded to the surface and said detector is integrated into said support.

35. Biosensor according to claim 30, wherein said collector molecules are covalently bonded to said surface.

36. Biosensor according to claim 30, wherein said detector is integrated into said support.

37. Biosensor according to claim 30, wherein said detector is a film is/are adhesively attached to said support.

38. Biosensor according to claim 30, wherein said detector(s) is/are arranged in a vicinity of said microchip, spaced a distance away from said microchip.

39. Biosensor according to claim 31, wherein said at least one detector is a photodiode that can also act as said excitation source at the same time.

40. Biosensor according to claim 30, wherein at least one type of collector molecule is immobilised on said surface in individual detection fields or in a form of a matrix or pattern.

41. Biosensor according to claim 30, wherein several types of collector molecules are immobilised on said surface.

42. Biosensor according to claim 40, wherein different collector molecules are immobilised on different detection fields or distinct positions of said matrix.

43. Biosensor according to claim 30, wherein said collector molecules are selected from the group consisting of single or double-stranded nucleic acids, nucleic acid analogues, haptenes, proteins, peptides, antibodies or their fragments, sugar structures, receptors, or ligands.

44. Biosensor according to claim 30, additionally comprising one or more elements from the group consisting of: a control unit, at least one amplifier, one or more signal transformers, one or more memory/storage units, one or more filters, an optics system, light guides, and one or more protective layers.

45. Biosensor according to claim 30, comprising a plurality of detectors.

46. Biosensor according to claim 45, whereby each detector is assigned to one field or one position of said matrix, preferably by being arranged below the field or position, and the size of a measurement surface area corresponds essentially to the field size.

47. Biosensor according to claim 30, wherein said collector molecules are arranged inside a base of a depression of said surface, whereby the base of the depression is recessed by at least 100 nm relative to said surface.

48. Method for detecting an analyte/collector complex by means of time-resolved luminescence using an optical biosensor in the form of a microchip, the biosensor comprising
(a) a support with a surface onto which at least one type of collector molecule is immobilised, and
(b) at least one detector that can detect light passing through the surface;
the method comprising the following steps:
(1) converting luminophores bound to the collector molecules and/or an analyte/collector complex into an excited state for an excitation time T₁,
(2) providing essentially no excitation for a die-away time T₂, and
(3) detecting emitted luminescent light with at least one detector for a time period T₃, and evaluating the emitted luminescent light to detect the luminophores bound to the collector molecules and/or the analyte/collector complex.

49. Method for detecting an analyte/collector complex by means of time-resolved luminescence using an optical biosensor in the form of a microchip, the biosensor comprising
(a) a support with a surface onto which at least one type of collector molecule is immobilised,
(b) at least one detector that can detect light passing through the surface, and
(c) at least one excitation source that can induce the emission of luminescent light;
the method comprising the following steps:
(1) converting luminophores bound to the collector molecules and/or an analyte/collector complex into an excited state for an excitation time T₁,
(2) providing essentially no excitation for a die-away time T₂, and
(3) detecting emitted luminescent light with at least one detector for a time period T₃, and evaluating the emitted luminescent light to detect the luminophores bound to the collector molecules and/or the analyte/collector complex.

50. Method according to claim 48, wherein in step (3) a plurality of analyte/collector complexes are detected in parallel by a parallel detection of luminescent light of different wavelengths.

51. Method according to claim 48, additionally comprising a step (4) comprising detecting luminescent light emitted at a wavelength different to a wavelength of light detected in step (3) for a subsequent time period T₄ and evaluating the luminescent light to detect a second complex.

52. Method according to claim 48, wherein an excitation takes place only in step (1).

53. Method according to claim 48, wherein steps (1) to (3) are performed several times.

54. Method according to claim 51, wherein steps (1) to (4) are performed several times.

55. Method according to claim 48, further comprising an initial step of bringing the collector molecules into contact with a sample that may or may not contain a ligand of the collector molecules as analyte, and, if necessary, washing the biosensor.

56. Method according to claim 48, wherein the ligand is labelled with a luminophore and detection takes place only when formation of an analyte/collector complex has taken place.

57. Method according to claim 48, wherein said time T₁ is 1 nanosecond to 2 milliseconds.

58. Method according to claim 48, wherein said time T₂ is 1 nanosecond to 500 microseconds.

59. Method according to claim 48, wherein said time T₃ is 5 nanoseconds to 10 milliseconds.

60. Method according to claim 48, wherein said luminophore is selected from the group consisting of: rare earth metals or actinide metals, in particular europium, terbium and samarium; semiconductors of Classes II-VI, III-V and IV, optionally doped, in particular CdSe, CdS or ZnS; and alkaline earth metal fluorides, especially CaF, and mixtures thereof.

61. Method according to claim 60, wherein said luminophore is used in the form of nanocrystals, beads or a chelate.

62. Method according to claim 48, for detecting a nucleic acid, nucleic acid analogues, a protein, peptide, haptene, antibody or fragment thereof, a sugar structure, a receptor or a ligand.

63. Method according to claim 48, wherein a biosensor according claim 30 is used.
